SEQUENCE LISTING (1 sequence)

SEQ ID NO: 1 - DNA, 1086 bp, ARTIFICIAL SEQUENCE
  SYNTHETIC OLIGONUCLEOTIDE
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatcccacgggctgcaggatgacccggaccttcaggccctcctgaagggcagccagcttctgaaggtgaagtccagctcgtggcgtagagaacgcttctacaagctacaggaggactgcaagaccatctggcaggaatctcgcaaggtcatgaggtccccggagtcgcagctgttctccatcgaggacattcaggaggtgcggatgggacaccgcacagaaggcctggagaaatttgccagagacatacccgaggaccgatgcttctccatcgtcttcaaggaccagcgcaataccctagacctcatcgccccgtctccagctgacgtccagcattgggtgcagggcctgcgcaagatcatcgaccgctcgggctccatggaccagcggcagaagtaa

(19) United States
(12) Patent Application Publication
(10) Pub. No.: US 2004/0253656 A1
(43) Pub. Date: Dec. 16, 2004

(54) Method for measuring inositol triphosphate

(76) Inventors: David Williams, Cardiff (GB); Peter James Tatnell, Cardiff (GB)

Correspondence Address: AMERSHAM BIOSCIENCES, PATENT DEPARTMENT, 800 CENTENNIAL AVENUE, PISCATAWAY, NJ, 08855, US

(21) Appl. No.: 10/865,310

(22) Filed: Jun. 10, 2004

(30) Foreign Application Priority Data
Jun. 30, 2003 (GB) 0315244.4
Jun. 11, 2003 (GB) 0313430.1

(51) Int. Cl. C12Q 1/68; G01N 33/53; G01N 33/537; G01N 33/543

(52) U.S. Cl. 435/7.92

(57) ABSTRACT

The invention relates to a homogeneous method for measuring the concentration of inositol 1,4,5-trisphosphate in a sample using a scintillation proximity assay. The invention also relates to a bead for use in a scintillation proximity assay which comprises a protein and a capture moiety.

### CROSS-REFERENCE TO RELATED APPLICATIONS

This application claims the benefit of patent application number 0315244.4 filed in Great Britain on Jun. 30, 2003 and patent application number 0313430.1 filed in Great Britain on Jun. 11, 2003; the entire disclosures of which are incorporated herein by reference in their entirety.

The present invention relates to a method and kit for measuring inositol 1,4,5-trisphosphate in a sample using a scintillation proximity assay.

### BACKGROUND OF THE INVENTION

Inositol 1,4,5-trisphosphate (hereinafter also referred to as ‘IP3’) is a secondary messenger which is produced by inositol phospholipid metabolism activated in response to extra-cellular stimuli such as hormones, growth factors, neurotransmitters and the like. The production of IP3 induces an increase in the intracellular concentration of calcium which plays a vital role in the signal transduction mechanism, and is involved in many cell functions across a diverse range of organisms. Thus, for example, IP3 controls many physiological functions such as fertilisation, blastogenesis, development and differentiation, cell growth, secretion, muscle contraction and cranial nerve functions in diverse organisms such as nematodes, insects, molluscs and mammals.

At the molecular level, receptor stimulation triggers hydrolysis of phosphatidylinositol bisphosphonate (PIP2) to diacylglycerol and inositol 1,4,5-trisphosphate (Downes et al. (1985) Mol. Mech. Trans Sign., 3-56). IP3 then activates specific intracellular receptor sites to release calcium from intracellular stores, such as the endoplasmic reticulum (Berridge et al. (1984), Nature, 312, 315-321), thereby controlling the activities of calcium-dependent proteins and enzymes.

The measurement of IP3 in cells and tissues is of considerable interest as it will enable a greater understanding of the role of this second messenger in a wide range of developmental and physiological processes and disorders. The control of IP3 levels within the cell is of particular interest to pharmaceutical and biotech companies in the development of new medicaments to treat disease. Interest also exists within the agrochemical sector for modulating IP3 levels to control insect (Raghu & Hasan (1995) Dev. Biol., 171, 564-77) and/or nematode (Walker et al. (2002) Mol. Biol Cell., 13, 1329-37), growth and development. There is therefore a need, within the pharmaceutical, biotechnology and agrochemical industries, for a high-throughput method or assay to measure IP3 levels following treatment of samples of cells, tissues or whole organisms with agonists, antagonists, inhibitors or enhancers. Ideally, such an assay could be in situ in nature, being used directly on cultured cells in the vessel in which they are being grown, following treatment with a test agent.

Traditionally the majority of methods to measure IP3 are non-homogeneous in nature and therefore require multiple steps, typical examples being affinity chromatography, PEG precipitation and filter binding assays. These methods are both time consuming and labour intensive, involving multiple steps which are prone to errors.

Examples of homogeneous methods or assays involve the use of luminescence proximity assays. This technique uses donor and acceptor beads; biological interactions bring the beads into close proximity generating a signal, which is amplified upon addition of a detection reagent. The reaction relies on a biotinylated IP3 analog and a GST-tagged IP3 binding protein. The biotinylated IP3 analog and GST-tagged IP3 binding protein are recognised by the streptavidin-donor and anti-GST conjugated acceptor beads. The beads are brought into close proximity and a signal is detected. Although this method is homogeneous, dedicated instrumentation is required.

Fluorescent methods also exist, such as fluorescence energy transfer (FRET) (Hamman et al. (2002) J Biomol Screen., 7, 45-55). In this particular application a GFP (Green fluorescent protein) is attached to a 170 amino acid protein obtained from an overexpressed Tec family kinase, containing a Plecktsrin Homology domain (hereinafter referred to as a ‘PH domain’). Homogeneous unilaminar vesicles were made that contained PIP3 (phosphatidtlyinositol trisphosphate) and octadecylrhodamine (OR), or a lipophilic FRET acceptor for GFP. Binding of the GFP-PH170 protein to the PIP3 in vesicles that contain OR results in a reduction in GFP fluorescence.

Grey et al. (Anal. Biochem (2003) 313, 234-245) have also demonstrated fluorescent-based methods for the detection of PIP2 and PIP3 using the GRP1 or TAPP1 PH domains in conjunction with biotin/streptavidin donor beads and GST/anti-GST acceptor beads.

Radioisotopic assays have been desribed for the detection of inositol phosphates. For example, Takenawa (WP1 Abstract Accession No. 1996-091675[10] and JP8000294A (Fujirebio KK 09-01-1996)) reports the use of a recombinant phospholipase C_{δ1} PH domain bound to sepharose beads to measure IP3 in test samples. The amount of tritium labelled analyte displaced from the beads is used as a measure of IP3 in the test sample in this non homogeneous assay.

Scintillation Proximity Assay (SPA) is a homogeneous radioisotopic assay. In a SPA, there is a solid phase (e.g., a bead or the bottom of a microplate) that is or contains within it a substance capable of fluorescing when stimulated by a β-particle that has been emitted by a weakly emitting β-isotope such as ³H or ¹²⁵I. The fluorescent substance is known as a scintillant or phosphor. The surface of the solid phase is such that it has an affinity for the particular analyte the assay is designed to detect. This can be done by modifying the surface of the solid so that it is coated with a receptor where the analyte is a substance that has an affinity for the receptor (e.g., a ligand of the receptor).

WO 97/49990 discloses a radioligand displacement assay for measuring PIP3 levels based upon alkaline hydrolysis of PIP3 to inositol tetrakisphospate (PIP4) which is then bound to a specific binding protein which lacks a PH domain.

WO 03/021220 describes a SPA for inositol phosphates where binding to the solid phase relies on a charge interaction between the negatively charged analyte and the positively charged solid phase. However, such an interaction may lead to problems of specificity due to interference from other negatively charged analytes.

WO 03/011901 postulates a SPA-based system as a screening assay where, for example, either the polypeptide or the phosphoinositide may be immobilised on the SPA beads and the ability of the test compound to disrupt the interaction between the polypeptide and the phosphoinositide may be measured. However, no detailed description of the assay or examples of its use are disclosed.

Accurate quantification of IP3 in tissues and cells requires thorough extraction in a suitable buffer that is free of interfering substances. Several documented methods are available to achieve this, for example, TCA precipitation followed by ether extraction. The sample is neutralised and the contaminating ether removed usually by drying under nitrogen. Alternative methods use acid extraction followed by treatment with sodium hydroxide to neutralise the sample. In addition the majority of these methods also require further downstream processing such as desalting, freeze-drying or spin chromatography.

The present invention seeks to address the above problems and to provide a homogeneous method for measuring IP3, particularly in tissues and cells following sample extraction, that is amenable to multiple sample or test analyses.

In another embodiment, the invention provides an in situ method for the extraction and measurement of IP3 from cultured cells that can be conducted directly in the vessel in which the cells have been grown. This is achieved using proprietary lysis reagents that have been optimised with the IP3 assay kit. This enables researchers to lyse cells and measure IP3 levels directly in the microplate, thus reducing time and avoiding losses that would be likely to occur as a result of the extra steps that are normally employed.

### SUMMARY OF THE INVENTION

According to the first aspect of the invention there is provided a homogeneous method for measuring the concentration of inositol 1,4,5-trisphosphate in a sample, the method comprising the steps of

a) incubating a protein comprising a Pleckstrin Homology Domain and a capture moiety with radioactively labelled inositol 1,4,5-trisphosphate to produce a protein-inositol 1,4,5-trisphosphate complex,

b) capturing the protein-inositol 1, 4, 5-trisphosphate complex on a solid phase that comprises a phosphor and a capture reagent that specifically binds to the capture moiety,

c) incubating the solid phase with the sample under conditions wherein labelled inositol 1,4,5-trisphosphate bound to the solid support is displaced by unlabelled inositol 1,4,5-trisphosphate present in the sample, and

d) detecting the amount of labelled inositol 1,4,5 trisphosphate remaining bound to the solid phase.

Pleckstrin Homology domains consist of regions of 100-120 amino acids found in numerous proteins involved in cell signalling (Haslam et al. (1993), Nature, 363, 309-310). The majority of PH domains appear to bind phosphoinositides, which may allow PH domain-containing proteins to respond to lipid messengers, for example by relocation to the cell membrane. The PLC_{δ1}/PH domain has an affinity for IP3 of 200 nM (Lemmon & Ferguson (2001) Biochem. Soc. Trans., 29, 377-384). PH domains are highly conserved, consisting of two orthagonal antiparallel β-sheets and a C-terminal amphiphilic α-helix (Hitoshi Yagisawa et al. (1998), J. Biol. Chem., 273, 417-424). U.S. Pat. No. 6,221,841 describes the cloning and characterisation of proteins containing PH domains, including the production of GST-fusion proteins which allow binding of the protein to a matrix. The high degree of specificity of the protein's PH domain to bind to PIP3 is demonstrated in this patent.

According to a second aspect of the present invention, there is provided a homogeneous method for measuring the effect a test agent has upon the concentration of inositol 1,4,5-trisphosphate in a sample comprising the steps of

a) contacting a protein comprising a Pleckstrin Homology Domain and a capture moiety with radioactively labelled inositol 1,4,5-trisphosphate to produce a protein-inositol 1,4,5-trisphosphate complex,

b) capturing the protein-inositol 1, 4, 5-trisphosphate complex on a solid phase that comprises a phosphor and a capture reagent that specifically binds to the capture moiety,

c) incubating the solid phase with the sample which has been treated with the test agent under conditions wherein labelled inositol 1,4,5-trisphosphate bound to the solid support is displaced by unlabelled inositol 1,4,5-trisphosphate present in the sample, and

d) detecting the amount of labelled inositol 1,4,5-trisphosphate remaining bound to the solid support and comparing this value with that obtained from a control sample which has not been treated with a test agent, any difference being indicative of the effect of the agent.

Suitably the value obtained from the control sample is already known prior to carrying out the method and may, for example, be stored on a database such as a digital computer.

A test agent may be, for example, any organic or inorganic compound such as a synthetic molecule or a natural product (e.g. peptide, oligonucleotide, hormone). Alternatively, the test agent may be an energy form such as light, heat or other forms of electro magnetic radiation. Suitably the test agent is an agonist, antagonist, inhibitor, or enhancer. As described herein, an agonist is any ligand (especially a drug or hormone) that binds to a receptor to alter the proportion that is in an active form to elicit a biological response. An antagonist is described herein as any ligand that results in the inverse response to an agonist while an inhibitor is any agent that blocks the biological response generated by the agonist. An enhancer is described herein as any agent that upregulates the biological response generated by the agonist.

Suitably, the amount of inositol 1,4,5-triphosphate in the method of the first or second aspect is determined by comparison to a standard curve based upon known concentrations of inositol 1,4,5-triphosphate. A typical standard curve according to the invention is shown in FIG. 1.

Preferably the sample is selected from the group consisting of organism, tissue and cell. Most preferably the sample is a cell. In order to analyse the intracellular concentrations of inositol 1,4,5-triphosphate the method preferably involves treating the cell with a lysis reagent prior to step b) of the first or second aspect of the invention and sequestering the reagent with a sequestrant. Preferably the lysis reagent is a detergent and the sequestrant is a cyclodextrin. Details of the lysis reaction and its subsequent inhibition by the sequestrant are described in European Patent Application 863402 (‘In-Situ Cell Extraction and Assay Method’).

Suitably, the detergent is a surface active agent which may be cationic, anionic, zwitterionic or non-ionic in nature. Examples of suitable detergents include dodecyl trimethyl ammonium bromide (DTAB); cetyl pyridinium chloride (CPC); benzethonium chloride (BZC); sodium dodecyl sulphate (SDS), and N-dodecyl-N,N-dimethyl-3-ammonio-1-propane sulphonate (DDAPS). DTAB, CPC and BZC are cationic surfactants; DDAPS is a zwitterionic surfactant and SDS is an anionic surfactant. Typical concentrations of detergent are in the range of 0.25-4% of the weight of the cell lysis fluid. In addition to lysing cells the detergent may also adversely affect the binding of the IP3 to the Pleckstrin Homology Domain and/or of the binding of the capture moiety to the capture reagent. The sequestrant is used to inhibit or annul that undesired adverse effect.

The sequestrant acts to prevent the detergent and any associated components bound thereto from adversely affecting a binding reaction between the IP3 and the Pleckstrin Homology Domain and/or the binding of the capture moiety to the capture reagent. The sequestrant may do this e.g. by chemically reacting with the detergent or by physically absorbing it. Preferred sequestrants are complex carbohydrate molecules such as cyclodextrins. Cyclodextrins are toroidal molecules consisting of 6, 7 or 8 glucose units (α-, β- and γ-cyclodextrin). The interior of the ring binds a hydrophobic tail of a molecule such as a surfactant. The resultant inclusion complex is generally formed with a 1:1 stoichiometry between surfactant and cyclodextrin. γ-Cyclodextrin and particularly α-cyclodextrin are preferred for use in this invention. Preferably enough sequestrant is used to be capable of sequestering or inactivating all cell lysis reagents present. Suitably the amount of sequestrant is from 0.5-10% by weight of the weight of the reaction mixture.

In a preferred embodiment, the method involves conducting the assay in a single vessel in which the cell is growing. The vessel may, for example, be the well of a microwell plate. Suitable microwell plates, or microtitre plates, are well known in the art and are commercially available from a range of suppliers (e.g. Greiner Labortechnik, Corning).

Suitably the solid phase is a bead suitable for use in a scintillation proximity assay. More suitably the bead comprises polyvinyl toluene or polystyrene.

Suitably, the solid phase is a coating on the base and/or side of a vessel. Preferably the vessel is a well of a microplate.

In a further aspect, the protein is selected from the group consisting of, for example, Pleckstrin, dynamin, Brutons tyrosine kinase and phospholipase C.

Suitably, the capture moiety and capture reagent are members of a specific binding pair. Preferably, the capture moiety/capture reagent is selected from the group consisting of antigen/antibody, biotin/steptavidin, biotin/avidin, GST/anti GST, His tags and maltose binding. More preferably, the capture moiety is biotin and the capture reagent is either streptavidin or avidin.

Suitably, the label is selected from the group consisting of ³H, ¹⁴C, ³²P, ³³P, ³⁵S and ¹²⁵I.

In a third aspect of the present invention, there is provided a kit for determining the concentration of inositol 1,4,5-trisphosphate in a sample comprising a protein comprising a Pleckstrin Homology Domain and a capture moiety, and a solid phase comprising a phosphor and a capture reagent that specifically binds to the capture moiety.

Suitably, the protein of the third aspect comprises a phospholipase C comprising a Pleckstrin Homology Domain and a biotin capture moiety; and the solid phase of the third aspect, which is suitable for use in a scintillation proximity assay, is coated with streptavidin or avidin. Suitably, the solid phase is eithera bead or the base/side of a vessel.

The kit of the third aspect may additionally comprise a lysis reagent, such as a detergent, and a sequestrant, such as cyclodextrin.

### BRIEF DESCRIPTION OF THE DRAWINGS

FIG. 1 illustrates the competitive displacement of labelled IP3 by unlabelled IP3.

FIG. 2 displays the results for the standard curves carried out in buffer alone or buffer plus lysis solutions 1 and 2 (n=3).

FIG. 3 shows the stimulatory effect carbochol has on cellular IP3 levels.

FIG. 4 shows Sequence ID Number 1.

### EXAMPLES

The following examples present certain preferred embodiments of the instant invention but are not intended to be illustrative of all embodiments. These examples should not be construed as limiting the appended claims and/or the scope of this invention.

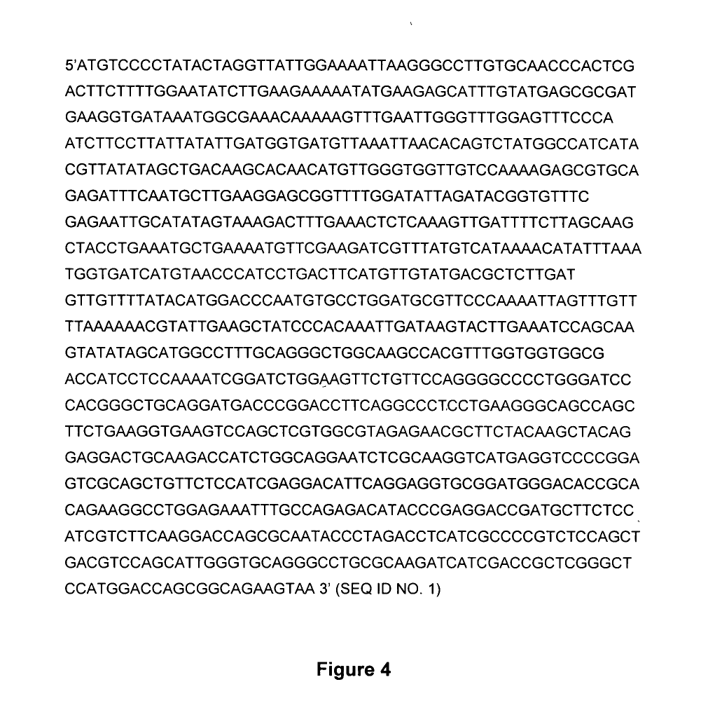

The human PLC_{δ1} PH domain was isolated by RT-PCR from RNA extracted from HeLa cells and cloned into pGEM-T (Promega). The initial amplicon was 664 bps in length and consisted of sequences that spanned from approximately 60 bps upstream of the initiator codon at met1 to 220 bps downstream of the C-terminal alpha helix of the PH domain (Sequence ID No.1; FIG. 4).

Oligo-nucleotide primers were designed to generate an amplicon (˜400 bps) that spanned sequences encoding from His11 to Lys 140 of the human PLC_{δ1} PH domain. Additional sequences were engineered to facilitate the subsequent cloning of each domain into the NdeI/BamHI and BamHI/XhoI sites of the expression vector pGEX-6P-1 (Amersham) Expression of the cDNA from pGEX-6P-1 derived constructs generates fusion proteins that possess a GST fusion partner at the N-terminus.

Soluble human proteins were produced in E. coli (BL21) by the addition of 1 mM IPTG (Isopropyl-β-D-thiogalactoside) at OD₆₀₀˜0.6. Bacterial cultures were induced for 3 hours. Cells were lysed using Novagens ‘bugbuster reagent’ in the presence of protease inhibitors (Roche Complete^{RTM}) and 1 mM β-mercaptoethanol. Cell debris was removed by centrifugation at 20,000 g/20 minutes and the 42 kDa PH GST linked protein was purified from the supernatant to near homogeneity using GST trap (Amersham) affinity columns. Fractions containing the protein of interest were combined and the buffer was exchanged to PSB by desalting (Sephadex columns, Amersham). The protein was quantified using Coommassie blue.

The protein was biotinylated at a ratio of 10 moles of biotin to 1 mole of protein using biotin NHS ester and rolled at room temperature for 45 mins, excess biotin was removed by desalting. Purity of the protein was determined by Coommassie blue staining following SDS-PAGE electrophoresis.

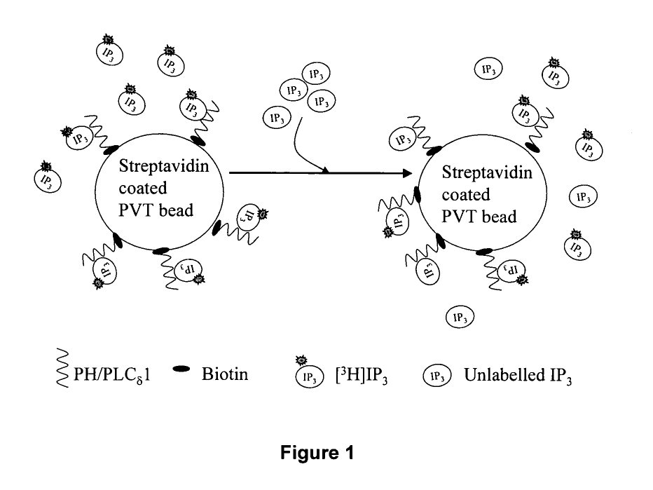

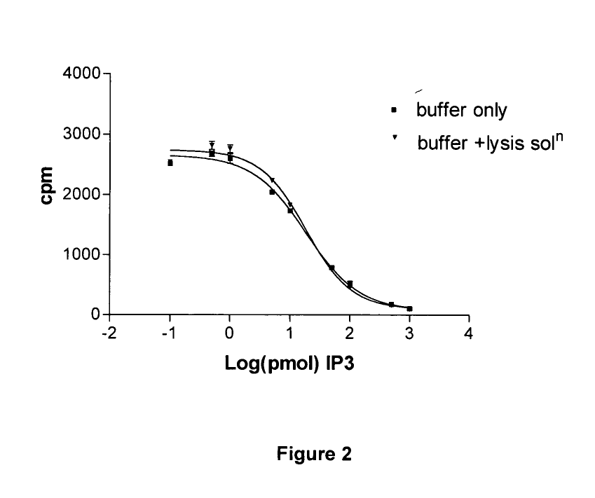

The competitive displacement of labelled IP3 by unlabelled material is illustrated in FIG. 1. The assay involves a phopholipase C recombinant protein containing a PH domain (PH/PLC_{δ}) that specifically binds IP3. The recombinant protein is biotinylated and captured using streptavidin coated polyvinyl toluene (PVT) beads. A signal is generated upon addition of [³H]labelled IP3. The assay is based on the competition between labelled [³H] IP3 and unlabelled IP3 in the standard or samples for binding to the PH domain. Quantification of unknown samples is determined by interpolation from a standard curve of known IP3 concentrations (e.g. FIG. 2).

In Vitro Assays

Standard curves were generated, by displacing labelled IP3, using a range of known concentrations of unlabelled IP3. Reactions were conducted in 96 well Corning NBS microplates in a total volume of 10011. The assays were carried out using 10 mMTris/1 M EDTA pH 7.0 containing 400 ng of the biotinylated GST-tagged PLC, protein, 10 nCi [³H] IP3 (spec activity 23.0 Ci/mmol, Amersham), and 1 mg of streptavidin coated PVT beads. Plates were incubated on ice for 2 hours and the plates then read using a Microbeta microplate scintillation counter. Standard curves were also run in the presence of dodecyl trimethyl ammonium bromide (DTAB; lysis reagent 1) and alpha-cyclodextrin (lysis reagent 2) at final concentrations of 0.3% lysis solution 1 and 2% lysis reagent 2 (FIG. 2).

Cellular Assays

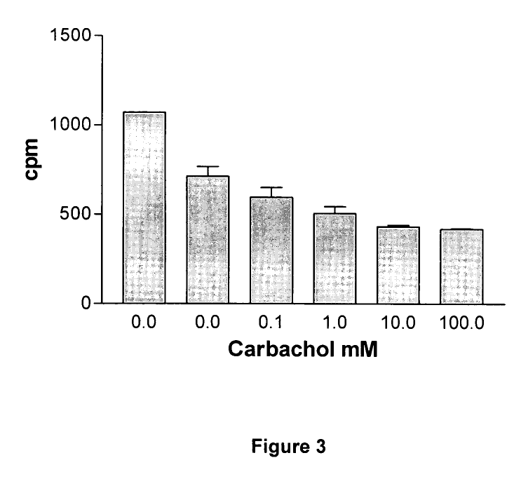

Cells (CHOM1; 2×10⁵) were seeded onto a 24 well tissue culture plate and grown overnight at 37° C., 95/5% (air/CO₂). Following overnight incubation, media were aspirated and the cells were washed 1× with PBS (phosphate buffered saline). Fresh media were added containing 20 mM LiCl (final concentration of lithium chloride) and the cells were incubated for 30 minutes at 37° C. in a humidified atmosphere (95/5%, air/CO₂). After 30 minutes Carbachol was added to give final concentrations ranging from 0.01 mM to 100 mM. The cells were incubated at 37° C., 95/5% (air/CO₂) for 1 minute and the media were aspirated. The cells were briefly washed using 1×PBS and the cells lysed using 10011 of 1% lysis reagent 1 containing 20 mM LiCl. The plate was incubated at room temperature for 30 minutes then centrifuged at 241 g for 4 minutes using an eppendorf 5804. The cell extract was transferred to an assay plate, 8011 of sequestrant added (lysis reagent 2, 10%) and finally the rest of the assay reagents added (see ‘in vitro assays’ above). The plate was incubated on ice for approximately 16 hours and the plate counted using a Microbeta microplate scintillation counter at 1 minute/well. The stimulatory effect on cellular levels of IP3 elicted by carbachol is shown in FIG. 3.

Those skilled in the art having the benefit of the teachings of the present invention as set forth above, can effect numerous modifications thereto. These modifications are to be construed as being encompassed within the scope of the present invention as set forth in the appended claims.

What is claimed is:

1. A homogeneous method for measuring the concentration of inositol 1,4,5-trisphosphate in a sample, the method comprising the steps of
a) incubating a protein comprising a Pleckstrin Homology Domain and a capture moiety with radioactively labelled inositol 1,4,5-trisphosphate to produce a protein-inositol 1,4,5-trisphosphate complex,
b) capturing said protein-inositol 1, 4, 5-trisphosphate complex on a solid phase that comprises a phosphor and a capture reagent that specifically binds to said capture moiety,
c) incubating said solid phase with said sample under conditions wherein labelled inositol 1,4,5-trisphosphate bound to the solid support is displaced by unlabelled inositol 1,4,5-trisphosphate present in the sample, and
d) detecting the amount of labelled inositol 1,4,5 trisphosphate remaining bound to the solid phase.

2. A homogeneous method for measuring the effect a test agent has upon the concentration of inositol 1,4,5-trisphosphate in a sample comprising the steps of
a) contacting a protein comprising a Pleckstrin Homology Domain and a capture moiety with radioactively labelled inositol 1,4,5-trisphosphate to produce a protein-inositol 1,4,5-trisphosphate complex,
b) capturing said protein-inositol 1, 4, 5-trisphosphate complex on a solid phase that comprises a phosphor and a capture reagent that specifically binds to said capture moiety,
c) incubating said solid phase with said sample which has been treated with said test agent under conditions wherein labelled inositol 1,4,5-trisphosphate bound to the solid state is displaced by unlabelled inositol 1,4,5-trisphosphate present in the sample, and
d) detecting the amount of labelled inositol 1,4,5-trisphosphate remaining bound to the solid support and comparing this value with that obtained from a control sample which has not been treated with a test agent, any difference being indicative of the effect of the agent.

3. The method of claim 2, wherein said value obtained from a control sample is already known.

4. The method of claim 1, wherein the amount of inositol 1,4,5-trisphosphate is determined by comparison to a standard curve based upon known concentrations of inositol 1,4,5-triphosphate.

5. The method of claim 1, wherein said sample is selected from the group consisting of organisms, tissues and cells.

6. The method of claim 5, further comprising lysing the organism, tissue or cell with a lysis reagent prior to step b) and sequestering said reagent with a sequestrant.

7. The method of claim 6, wherein the lysis reagent is a detergent and the sequestrant is a cyclodextrin.

8. The method of claim 6, wherein said sample is a cell and said method is conducted in a single vessel in which said cell is growing.

9. The method of claim 1, wherein said solid phase is a bead.

10. The method of claim 9, wherein said bead comprises polyvinyl toluene or polystyrene.

11. The method of claim 1, wherein said solid phase is a coating on the base and/or side of a vessel.

12. The method of claim 11, wherein said vessel is a well of a microplate.

13. The method of claim 1, wherein the protein is selected from the group consisting of Pleckstrin, dynamin, Bruton's tyrosine kinase and phospholipase C.

14. The method of claim 1, wherein the capture moiety and capture reagent are members of a specific binding pair.

15. The method of claim 14, wherein the capture moiety and capture reagent are selected from the group consisting of antigen/antibody, biotin/steptavidin, biotin/avidin, GST/anti-GST tag, His tag and maltose binding.

16. The method of claim 1, wherein the capture moiety is biotin and the capture reagent is either streptavidin or avidin.

17. The method of claim 1, wherein said radioactive label is selected from the group consisting of ³H, ¹⁴C ³²P, ³³P, ³⁵S and ¹²⁵I.

18. The method of claim 2, wherein the test agent is an inhibitor, agonist, antagonist or enhancer.

19. A kit for determining the concentration of inositol 1,4,5-trisphosphate in a sample comprising
a protein comprising a Pleckstrin Homology Domain and a capture moiety, and
a solid phase comprising a phosphor and a capture reagent that specifically binds to said capture moiety.

20. The kit of claim 19, wherein said protein is a phospholipase C protein, said capture moiety is biotin and said capture reagent is streptavidin or avidin.

21. The kit of claim 20, wherein the solid phase is either a bead or the base or side of a vessel.
